(19) United States
(12) Patent Application Publication
(10) Pub. No.: US 2021/0000376 A1
(43) Pub. Date: Jan. 7, 2021

(54) ELECTRONIC DEVICE FOR ACQUIRING BIOSIGNALS AND OPERATION METHOD THEREFOR

(71) Applicant: Samsung Electronics Co., Ltd., Suwon-si, Gyeonggi-do (KR)

(72) Inventors: Suho LEE, Suwon-si (KR); Younghyun KIM, Suwon-si (KR); Seungeun LEE, Suwon-si (KR)

(21) Appl. No.: 16/969,532

(22) PCT Filed: Nov. 20, 2018
(86) PCT No.: PCT/KR2018/014290

§ 371 Date: Aug. 12, 2020

(30) Foreign Application Priority Data
Feb. 12, 2018 (KR) 10-2018-0016950

(51) Int. Cl. A61B 5/053; A61B 5/0402; A61B 5/00; A61B 5/0476; A61B 5/0496; A61B 5/0488

(52) U.S. Cl. CPC A61B 5/053; A61B 5/0402; A61B 5/4872; A61B 5/0488; A61B 5/7225; A61B 5/0496; A61B 5/0476

(57) ABSTRACT

An electronic device according to various embodiments of the present invention comprises: a sensor module; a first electrode unit connected to a first channel of the sensor module; and a second electrode unit connected to a second channel of the sensor module, and may be configured to: measure, through the sensor module, a first impedance between the first electrode unit and a first portion of a user's body contacting the first electrode unit, and a second impedance between the second electrode unit and a second portion of the user's body contacting the second electrode unit; adjust, through the sensor module, an impedance corresponding to each of the first channel and the second channel on the basis of the first impedance and the second impedance so that a difference between the first impedance and the second impedance is within a specified range; and obtain a biometric signal of the user through the sensor module while the impedance of the first channel and the second channel are adjusted.

### CROSS-REFERENCE TO RELATED APPLICATIONS

This application is a National Phase Entry of PCT International Application No. PCT/KR2018/014290, which was filed on Nov. 20, 2018 and claims priority to Korean Patent Application No. 10-2018-0016950, which was filed on Feb. 12, 2018 in the Korean Intellectual Property Office, the contents of which are incorporated herein by reference.

### BACKGROUND

### 1. Field

Various embodiments of the present invention relate to electronic devices obtaining biometric signals and methods for operating the same.

### 2. Description of the Related Art

More and more services and additional functions are provided through wearable electronic devices, smartphones or other portable electronic devices. To meet the needs of various users and raise use efficiency of electronic devices, communication service carriers or device manufacturers are jumping into competitions to develop electronic devices with differentiated and diversified functionalities.

As electronic devices come up with better capability, various biometric recognition techniques are being applied to electronic devices. Users may obtain and be given biometric information, body information, or health information using various biometric technologies applied to electronic devices.

As such, for portable electronic devices adopting various biometric technologies, accurate measurement of biometric signals may be of significance.

### SUMMARY

Biometric signals may be obtained via a plurality of electrodes included in an electronic device. The user may obtain biometric signals by bringing his/her body portion in contact with a plurality of electrodes included in the electronic device. If the user's body portion contacts the plurality of electrodes included in the electronic device, a touch impedance or contact impedance may occur in the area of contact. The contact impedance may add noise to the biometric signal obtained via the electrode.

The biometric signals obtained via the plurality of electrodes included in the electronic device have a low signal strength or magnitude, and the electronic device may amplify the biometric signals for analysis. If the biometric signal is amplified, the noise mixed with the biometric signal may be amplified as well.

To remove the noise, a high-performance amplifier may be used. However, use of a high-performance amplifier may be unsuitable for commercialization due to cost issues.

Or, the noise may be eliminated by reducing the magnitude of the contact impedance itself. However, this way requires use of larger electrodes, rendering it difficult to slim down the product.

According to various embodiments of the present invention, there may be provided an electronic device capable of removing noise, originating from contact impedance, from biometric signals by reducing the difference in contact impedance between a first electrode unit and a second electrode unit and a method for operating the electronic device.

According to various embodiments of the present invention, an electronic device comprises a sensor module, a first electrode unit connected with a first channel of the sensor module, a second electrode unit connected with a second channel of the sensor module, and a processor configured to measure, via the sensor module, a first impedance between the first electrode unit and a first portion of a user's body contacting the first electrode unit and a second impedance between the second electrode unit and a second portion of the user's body contacting the second electrode unit, adjust, via the sensor module, impedances corresponding to the first channel and the second channel, respectively, so that a difference between the first impedance and the second impedance meets a designated range, based on the first impedance and the second impedance, and obtain, via the sensor module, a biometric signal of the user, with the impedances of the first channel and the second channel adjusted.

According to various embodiments of the present invention, a method for operating an electronic device comprises measuring a first impedance between a first electrode unit connected with a first channel of a sensor module included in the electronic device and a first portion of a user's body contacting the first electrode unit, determining a second impedance between a second electrode unit connected with a second channel of the sensor module and a second portion of the user's body contacting the second electrode unit, adjusting impedances corresponding to the first channel and the second channel, respectively, so that a difference between the first impedance and the second impedance meets a designated range, based on the first impedance and the second impedance, and obtaining, via the sensor module, a biometric signal of the user, with the impedances of the first channel and the second channel adjusted.

According to various embodiments of the present invention, an electronic device is capable of efficiently removing noise, originating from contact impedance, from biometric signals by reducing the difference in contact impedance between a first electrode unit and a second electrode unit.

### BRIEF DESCRIPTION OF THE DRAWINGS

A more complete appreciation of the disclosure and many of the attendant aspects thereof will be readily obtained as the same becomes better understood by reference to the following detailed description when considered in connection with the accompanying drawings, wherein:

FIG. 1 is a view illustrating an electronic device in a network environment according to various embodiments;

FIG. 2 is a view illustrating a structure of an electronic device according to various embodiments of the present invention;

FIG. 3 is a block diagram illustrating operations of an electronic device according to various embodiments of the present invention;

FIG. 4 is a block diagram illustrating operations of an electronic device according to various embodiments of the present invention;

FIG. 5 is a block diagram illustrating operations of an electronic device according to various embodiments of the present invention;

FIG. 6 is a block diagram illustrating operations of an electronic device according to various embodiments of the present invention;

FIGS. 7 and 8 are views illustrating a biometric signal, from which noise caused by contact impedance has not been removed, and a noise-removed biometric signal, according to various embodiments of the present invention;

FIG. 9 is a flowchart illustrating operations of an electronic device according to various embodiments of the present invention;

FIG. 10 is a flowchart illustrating operations of an electronic device according to various embodiments of the present invention;

FIG. 11 is a flowchart illustrating operations of an electronic device according to various embodiments of the present invention;

FIG. 12 is a flowchart illustrating operations of an electronic device according to various embodiments of the present invention; and

FIGS. 13A to 13D are views illustrating a user interface showing the operation of measuring a measurement by an electronic device according to various embodiments of the present invention.

### DETAILED DESCRIPTION

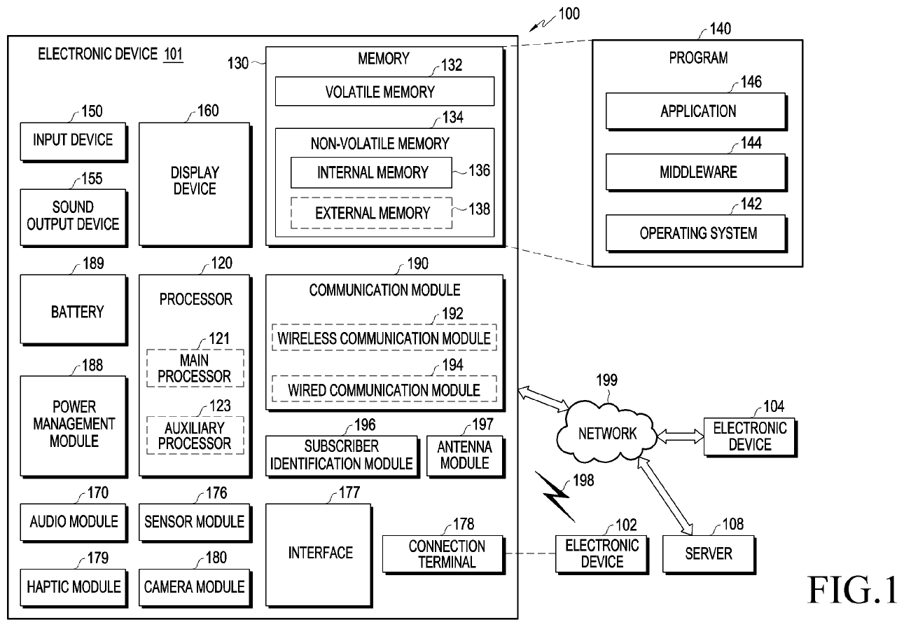

FIG. 1 is a block diagram illustrating an electronic device 101 in a network environment 100 according to various embodiments. Referring to FIG. 1, the electronic device 101 in the network environment 100 may communicate with an electronic device 102 via a first network 198 (e.g., a short-range wireless communication network), or an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, a memory 130, an input device 150, a sound output device 155, a display device 160, an audio module 170, a sensor module 176, an interface 177, a haptic module 179, a camera module 180, a power management module 188, a battery 189, a communication module 190, a subscriber identification module 196, and an antenna module 197. In some embodiments, the electronic device 101 may exclude at least one (e.g., the display device 160 or the camera module 180) of the components or add other components. In some embodiments, some components may be implemented to be integrated together, e.g., as if the sensor module 176 (e.g., a fingerprint sensor, an iris sensor, or an illuminance sensor) is embedded in the display device (160) (e.g., a display).

The processor 120 may drive, e.g., software (e.g., a program 140) to control at least one other component (e.g., a hardware or software component) of the electronic device 101 connected with the processor 120 and may process or compute various data. The processor 120 may load and process a command or data received from another component (e.g., the sensor module 176 or the communication module 190) on a volatile memory 132, and the processor 120 may store resultant data in a non-volatile memory 134. According to an embodiment, the processor 120 may include a main processor 121 (e.g., a central processing unit (CPU) or an application processor), and additionally or alternatively, an auxiliary processor 123 (e.g., a graphics processing unit (GPU), an image signal processor, a sensor hub processor, or a communication processor) that is operated independently from the main processor 121 and that consumes less power than the main processor 121 or is specified for a designated function. Here, the auxiliary processor 123 may be operated separately from or embedded in the main processor 121.

In such case, the auxiliary processor 123 may control at least some of functions or states related to at least one (e.g., the display device 160, the sensor module 176, or the communication module 190) of the components of the electronic device 101, instead of the main processor 121 while the main processor 121 is in an inactive (e.g., sleep) state or along with the main processor 121 while the main processor 121 is an active state (e.g., performing an application). According to an embodiment, the auxiliary processor 123 (e.g., an image signal processor or a communication processor) may be implemented as part of another component (e.g., the camera module 180 or the communication module 190) functionally related to the auxiliary processor 123. The memory 130 may store various data used by at least one component (e.g., the processor 120 or sensor module 176) of the electronic device 101, e.g., software (e.g., the program 140) and input data or output data for a command related to the software. The memory 130 may include the volatile memory 132 or the non-volatile memory 134.

The program 140, as software stored in the memory 130, may include, e.g., an operating system (OS) 142, middleware 144, or an application 146.

The input device 150 may be a device for receiving a command or data, which is to be used for a component (e.g., the processor 120) of the electronic device 101, from an outside (e.g., a user) of the electronic device 101. The input device 1150 may include, e.g., a microphone, a mouse, or a keyboard.

The sound output device 155 may be a device for outputting sound signals to the outside of the electronic device 101. The sound output device 1155 may include, e.g., a speaker which is used for general purposes, such as playing multimedia or recording and playing, and a receiver used for call receiving purposes only. According to an embodiment, the receiver may be formed integrally or separately from the speaker.

The display 160 may be a device for visually providing information to a user of the electronic device 101. The display device 2660 may include, e.g., a display, a hologram device, or a projector and a control circuit for controlling the display, hologram device, or projector. According to an embodiment, the display device 160 may include touch circuitry or a pressure sensor capable of measuring the strength of a pressure for a touch.

The audio module 170 may convert a sound into an electrical signal and vice versa. According to an embodiment, the audio module 170 may obtain a sound through the input device 150 or output a sound through the sound output device 155 or an external electronic device (e.g., an electronic device 102 (e.g., a speaker or a headphone) wiredly or wirelessly connected with the electronic device 101.

The sensor module 176 may generate an electrical signal or data value corresponding to an internal operating state (e.g., power or temperature) or external environmental state of the electronic device 101. The sensor module 176 may include, e.g., a gesture sensor, a gyro sensor, an atmospheric pressure sensor, a magnetic sensor, an acceleration sensor, a grip sensor, a proximity sensor, a color sensor, an infrared (IR) sensor, a bio sensor, a temperature sensor, a humidity sensor, or an illuminance sensor.

The interface 177 may support a designated protocol enabling a wired or wireless connection with an external electronic device (e.g., the electronic device 102). According to an embodiment, the interface 177 may include a high definition multimedia interface (HDMI), a universal serial bus (USB) interface, a secure digital (SD) card interface, or an audio interface.

A connecting terminal 178 may include a connector, e.g., a HDMI connector, a USB connector, an SD card connector, or an audio connector (e.g., a headphone connector), which is able to physically connect the electronic device 101 with an external electronic device (e.g., the electronic device 102).

The haptic module 179 may convert an electrical signal into a mechanical stimulus (e.g., a vibration or motion) or electrical stimulus which may be recognized by a user via his tactile sensation or kinesthetic sensation. The haptic module 179 may include, e.g., a motor, a piezoelectric element, or an electric stimulator.

The camera module 180 may capture a still image or moving images. According to an embodiment, the camera module 180 may include one or more lenses, an image sensor, an image signal processor, or a flash.

The power management module 188 may be a module for managing power supplied to the electronic device 101. The power management module 188 may be configured as at least part of, e.g., a power management integrated circuit (PMIC).

The battery 189 may be a device for supplying power to at least one component of the electronic device 101. The battery 189 may include, e.g., a primary cell which is not rechargeable, a secondary cell which is rechargeable, or a fuel cell.

The communication module 190 may support establishing a wired or wireless communication channel between the electronic device 101 and an external electronic device (e.g., the electronic device 102, the electronic device 104, or the server 108) and performing communication through the established communication channel. The communication module 190 may include one or more communication processors that are operated independently from the processor 120 (e.g., an application processor) and supports wired or wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of the wireless communication module 192 and the wired communication module 194 may be used to communicate with an external electronic device through a first network 198 (e.g., a short-range communication network, such as Bluetooth, wireless-fidelity (Wi-Fi) direct, or infrared data association (IrDA)) or a second network 199 (e.g., a long-range communication network, such as a cellular network, the Internet, or a communication network (e.g., LAN or wide area network (WAN)). The above-enumerated types of communication modules 190 may be implemented in a single chip or individually in separate chips.

According to an embodiment, the wireless communication module 192 may differentiate and authenticate the electronic device 101 in the communication network using user information stored in the subscriber identification module 196.

The antenna module 197 may include one or more antennas for transmitting or receiving a signal or power to/from an outside. According to an embodiment, the communication module 190 (e.g., the wireless communication module 192) may transmit or receive a signal to/from an external electronic device through an antenna appropriate for a communication scheme.

Some of the above-described components may be connected together through an inter-peripheral communication scheme (e.g., a bus, general purpose input/output (GPIO), serial peripheral interface (SPI), or mobile industry processor interface (MIPI)), communicating signals (e.g., commands or data) therebetween.

According to an embodiment, commands or data may be transmitted or received between the electronic device 101 and the external electronic device 104 via the server 108 coupled with the second network 199. Each of the electronic devices 102 and 104 may be a device of a same type as, or a different type, from the electronic device 101. According to an embodiment, all or some of operations executed on the electronic device 101 may be run on one or more other external electronic devices. According to an embodiment, when the electronic device 101 should perform a certain function or service automatically or at a request, the electronic device 101, instead of, or in addition to, executing the function or service on its own, may request an external electronic device to perform at least some functions associated therewith. The external electronic device (e.g., electronic devices 102 and 104 or server 106) may execute the requested functions or additional functions and transfer a result of the execution to the electronic device 101. The electronic device 101 may provide a requested function or service by processing the received result as it is or additionally. To that end, a cloud computing, distributed computing, or client-server computing technology may be used, for example.

The electronic device according to various embodiments may be one of various types of electronic devices. The electronic devices may include at least one of, e.g., a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the disclosure and the terms used therein are not intended to limit the techniques set forth herein to particular embodiments and that various changes, equivalents, and/or replacements therefor also fall within the scope of the disclosure. The same or similar reference denotations may be used to refer to the same or similar elements throughout the specification and the drawings. It is to be understood that the singular forms “a,” “an,” and “the” include plural references unless the context clearly dictates otherwise. As used herein, the term “A or B,” “at least one of A and/or B,” “A, B, or C,” or “at least one of A, B, and/or C” may include all possible combinations of the enumerated items. As used herein, the terms “1st” or “first” and “2nd” or “second” may modify corresponding components regardless of importance and/or order and are used to distinguish a component from another without limiting the components. It will be understood that when an element (e.g., a first element) is referred to as being (operatively or communicatively) “coupled with/to,” or “connected with/to” another element (e.g., a second element), it can be coupled or connected with/to the other element directly or via a third element.

As used herein, the term “module” includes a unit configured in hardware, software, or firmware and may interchangeably be used with other terms, e.g., “logic,” “logic block,” “part,” or “circuit.” A module may be a single integral part or a minimum unit or part for performing one or more functions. For example, the module may be configured in an application-specific integrated circuit (ASIC).

Various embodiments as set forth herein may be implemented as software (e.g., the program 140) containing commands that are stored in a machine (e.g., computer)-readable storage medium (e.g., an internal memory 136) or an external memory 138. The machine may be a device that may invoke a command stored in the storage medium and may be operated as per the invoked command. The machine may include an electronic device (e.g., the electronic device 101) according to embodiments disclosed herein. When the command is executed by a processor (e.g., the processor 120), the processor may perform a function corresponding to the command on its own or using other components under the control of the processor. The command may contain a code that is generated or executed by a compiler or an interpreter. The machine-readable storage medium may be provided in the form of a non-transitory storage medium. Here, the term “non-transitory” simply means that the storage medium does not include a signal and is tangible, but this term does not differentiate between where data is semipermanently stored in the storage medium and where data is temporarily stored in the storage medium.

According to an embodiment, a method according to various embodiments of the disclosure may be included and provided in a computer program product. The computer program products may be traded as commodities between sellers and buyers. The computer program product may be distributed in the form of a machine-readable storage medium (e.g., a compact disc read only memory (CD-ROM)) or online through an application store (e.g., Playstore™). When distributed online, at least part of the computer program product may be temporarily generated or at least temporarily stored in a storage medium, such as the manufacturer's server, a server of the application store, or a relay server.

According to various embodiments, each component (e.g., a module or program) may be configured of a single or multiple entities, and the various embodiments may exclude some of the above-described sub components or add other sub components. Alternatively or additionally, some components (e.g., modules or programs) may be integrated into a single entity that may then perform the respective (pre-integration) functions of the components in the same or similar manner. According to various embodiments, operations performed by modules, programs, or other components may be carried out sequentially, in parallel, repeatedly, or heuristically, or at least some operations may be executed in a different order or omitted, or other operations may be added.

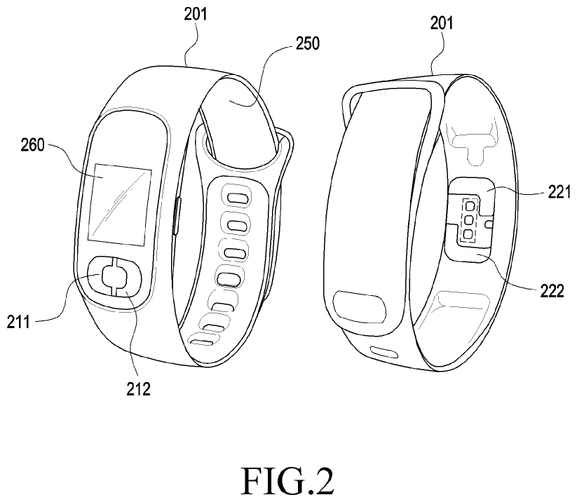

FIG. 2 is a view illustrating a structure of an electronic device according to various embodiments of the present invention.

Referring to FIG. 2, an electronic device 201 may be implemented in substantially the same or similar manner to the electronic device 101 described above in connection with FIG. 1. For example, the electronic device 201 may include at least some of the components of the electronic device 101 of FIG. 1. For example, the electronic device 201 may be implemented as a wearable electronic device that may be coupled to a portion of the user's body.

According to various embodiments, the electronic device 201 may include electrodes 211, 212, 221, and 222 and a display 260 (e.g., the display device 160 of FIG. 1). The electronic device 201 may include a housing that includes a first surface facing in a first direction, a second surface facing in a second direction, which is opposite to the first direction, and a side surface surrounding at least a portion of a space between the first surface and the second surface. For example, the first surface of the housing may mean a surface including a first electrode 211 and a second electrode 212, and the second surface of the housing may mean a surface including a third electrode 221 and a fourth electrode 222.

According to various embodiments, the electrodes 211, 212, 221, and 222 may be implemented as conductive members through which current may flow. For example, the electrodes 211, 212, 221, and 222 may be implemented as low-resistance conductive members (stainless steel, silver, and/or gold). The electrodes 211, 212, 221, and 222 may be formed in various shapes or sizes.

According to various embodiments, the electrodes 211, 212, 221, and 222 may be exposed to the outside through at least a portion of the housing constituting the electronic device 201. For example, at least one of the first electrode 211 and the second electrode 212 may be exposed to the outside through at least a portion of the first surface of the housing constituting the electronic device 201. Further, at least one of the third electrode 221 and the fourth electrode 222 may be exposed to the outside through at least a portion of the second surface of the housing constituting the electronic device 201.

According to various embodiments, at least one electrode included in the electronic device 201 may be present on the first or second surface of the electronic device 201 or a portion of the housing, except for the first and second surfaces. For example, the electronic device 201 may be configured to include all of the four electrodes 211, 212, 221, and 222 on the first or second surface of the housing. Further, the electronic device 201 may be configured to include all of the four electrodes 211, 212, 221, and 222 on a portion of the housing except for the first and second surfaces.

Although in the illustration of FIG. 2, the number, shape, size, and position of the electrodes 211, 212, 221, and 222 are designated for ease of description, the number, shape, size, and position of the electrodes 211, 212, 221, and 222 are not limited thereto but may be varied.

According to various embodiments, at least one of the electrodes 211, 212, 221, and 222 may be electrically connected with at least one biometric sensor (e.g., the biometric module 176 of FIG. 1) provided in the electronic device 101 and be used to obtain the user's biometric information, body information, or health information. For example, at least one of the electrodes 211, 212, 221, and 222 may be used to measure the bioelectric impedance analysis (BIA) via the biometric sensor provided in the electronic device 101 and to measure the user's body fat percentage. Further, at least one of the electrodes 211, 212, 221, and 222 may be used to measure the electrocardiogram (ECG) via the biometric sensor provided in the electronic device 101 and to measure the user's electrocardiogram. Further, at least one of the electrodes 211, 212, 221, and 222 may be used to measure the galvanic skin response (GSR) via the biometric sensor provided in the electronic device 101 and to measure (or calculate) the user's skin resistance and/or skin hydration. Meanwhile, the biometric information measurable using at least one of the electrodes 211, 212, 221, and 222 is merely an example and, without limitations thereto, the present invention may be implemented in other various forms.

According to various embodiments, at least one of the electrodes 211, 212, 221, and 222 may be electrically connected with a charging circuit provided in the electronic device 101. Here, the charging circuit may mean a circuit including a power management module (e.g., the power management module 188 of FIG. 1) and/or a battery (e.g., the battery 189 of FIG. 1). Or, the charging circuit may mean a circuit for electrically connecting at least one component included in the electronic device 101 with a power management module (e.g., the power management module 188) or a battery (e.g., the battery 189 of FIG. 1).

According to various embodiments, the charging circuit may be used to charge the battery (e.g., the battery 189 of FIG. 1) of the electronic device 101. For example, the charging circuit of the electronic device 101 may be physically or electrically connected with an external device (e.g., a charger or cradle) through at least one of the electrodes 211, 212, 221, and 222 exposed to the outside. The charging circuit of the electronic device 101 may be fed power via at least one electrode connected with the external device among the electrodes 211, 212, 221, and 222.

According to various embodiments, the electronic device 201 may generate the user's biometric information, body information, or health information based on a signal (e.g., a biometric signal) received via at least one of the electrodes 211, 212, 221, and 222. Further, the electronic device 201 may store the user's biometric information, body information, or health information in a memory (e.g., the memory 130 of FIG. 1).

According to various embodiments, the electronic device 201 may determine contact impedances (or contact impedance values) that occur in the areas where the electrodes 211, 212, 221, and 222 touch or contact the user's body. The electronic device 201 may perform an operation for reducing an error in the biometric signal generated due to the contact impedance.

According to various embodiments, the display 260 (e.g., the display device 160 of FIG. 1) may display information related to the operation or state of the electronic device 201. The display 260 may display the user's biometric information, body information, or health information based on a signal (e.g., a biometric signal) received via at least one of the electrodes 211, 212, 221, and 222.

According to various embodiments, the electronic device 101 may display the user's biometric information, body information, or health information on a display of an external device (e.g., the external device 102 of FIG. 1), based on a signal (e.g., a biometric signal) received via at least one of the electrodes 211, 212, 221, and 222.

According to various embodiments, the electronic device 201 may provide the user with some of the signals generated from the electronic device 201, in various forms (e.g., light (LED), sounds, or vibrations), via an output interface (e.g., the sound output device 155 or haptic module 179 of FIG. 1) of the electronic device 201.

According to various embodiments, the electronic device 201 may include a coupling member 250 that is connected with a portion of the housing and detachably fastens the electronic device 201 to a portion of the user's body.

According to various embodiments, the electronic device 201 may analyze, through an external device (e.g., the external device 102 of FIG. 1), the signals (e.g., biometric signals) received via the electrodes 211, 212, 221, and 222. For example, the electronic device 201 may transmit the signals received via the electrodes 211, 212, 221, and 222 to the external device 102 via the communication module (e.g., the communication module 190 of FIG. 1) of the electronic device 201. The external device 102 may analyze the signal received from the electronic device 201 and transmit the analyzed information to the electronic device 201. The electronic device 201 may provide content corresponding to the user's biometric information, body information, or health information, via the display 260, based on the information received from the external device 102.

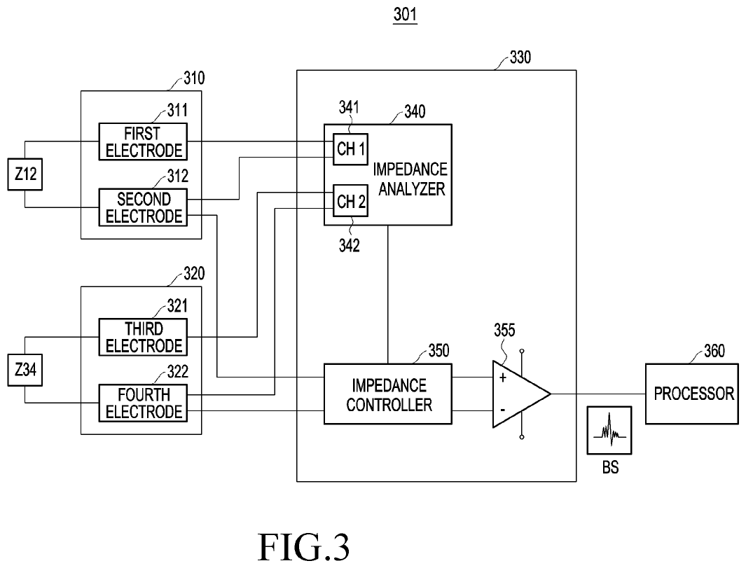

FIG. 3 is a block diagram illustrating operations of an electronic device according to various embodiments of the present invention.

Referring to FIG. 3, an electronic device 301 (e.g., the electronic device 101 of FIG. 1) may include a first electrode unit 310, a second electrode unit 320, a sensor module 330 (e.g., the sensor module 176 of FIG. 1), and a processor 360 (e.g., the processor 120 of FIG. 1). For example, the electronic device 301 may be implemented to be substantially the same or similar to the electronic devices 101 and 102 of FIGS. 1 and 2.

The first electrode unit 310 may include a first electrode 311 (e.g., the electrode 211 of FIG. 2) and a second electrode 312 (e.g., the electrode 212 of FIG. 2). For example, the first electrode unit 310 may be positioned on a first surface (or second surface) of the housing of the electronic device 201 of FIG. 2. In other words, the first electrode 311 and the second electrode 312 may be electrodes positioned on the first surface (or second surface) of the housing.

The first electrode unit 310 may be connected with a first channel CH1 of the sensor module 330. For example, the first electrode 311 and the second electrode 312 may be connected with the first channel CH1 of the sensor module 330 via different paths.

The first electrode unit 310 may contact at least a portion of the user's body. For example, the first electrode unit 310 may contact a first portion of the user's body. Further, the first electrode unit 310 may obtain a signal indicating the impedance for the first portion (e.g., a portion including the surface of the first portion) of the user's body. Further, the first electrode unit 310 may obtain the user's biometric signal via the first portion of the user's body.

The second electrode unit 320 may include a third electrode 321 (e.g., the electrode 221 of FIG. 2) and a fourth electrode 322 (e.g., the electrode 222 of FIG. 2). For example, the second electrode unit 320 may be positioned on a second surface (or first surface) of the housing of the electronic device 201 of FIG. 2. In other words, the third electrode 321 and the fourth electrode 322 may be electrodes positioned on the second surface (or first surface) of the housing.

The second electrode unit 320 may be connected with a second channel CH2 of the sensor module 330. For example, the third electrode 321 and the fourth electrode 322 may be connected with the second channel CH2 of the sensor module 330 via different paths.

The second electrode unit 320 may contact a second portion of the user's body. For example, the second portion of the user's body, which contacts the second electrode unit 320, may be identical to or different from the first portion of the user's body, which contacts the first electrode unit 310. The second electrode unit 320 may receive a signal indicating the impedance for the area contacted via the user's body portion. Further, the second electrode unit 320 may obtain the user's biometric signal via the second portion of the user's body.

According to an embodiment, the first electrode unit 310 may contact the first portion of the user's body, causing a contact impedance (or touch impedance). Further, the second electrode unit 320 may contact the second portion of the user's body, causing a contact impedance. At this time, the contact impedance may be determined by at least one of the metal, area of contact, contacted portion, temperature, humidity, foreign bodies, and skin type.

The sensor module 330 may sense or obtain the user's biometric or health-related signal. The sensor module 330 may be implemented in the same or similar manner to the sensor module 176 of FIG. 1. For example, the sensor module 330 may sense a signal related to at least one of the user's body fat (BIA), electrocardiogram (ECG), skin resistance (GSR), electromyogram (EMG), electroencephalogram (EEG), and electrooculogram (EOG).

The processor 360 may control the overall operation of the electronic device 301.

According to an embodiment, the processor 360 may obtain a signal related to the user's body via the first electrode unit 310 and the second electrode unit 320 and generate the user's biometric signal (BS) based on the obtained signal. For example, if there is a request from the user (e.g., if an application related to biometric information measurement runs), the processor 360 may obtain a body-related signal and generate the user's biometric signal (BS) based on the obtained signal. For example, the biometric signal (BS) may mean a signal indicating the user's biometric information obtained from the user's body. The processor 360 may analyze the biometric signal (BS) and provide biometric information to the user. For example, the biometric information may include information about the user's body fat (BIA), electrocardiogram (ECG), skin resistance (GSR), electromyogram (EMG), electroencephalogram (EEG), and electrooculogram (EOG).

According to an embodiment, the processor 360 may display the biometric information on a display (e.g., the display 260 of FIG. 2). Further, the processor 360 may store the biometric information in a memory (e.g., the memory 130 of FIG. 1).

According to an embodiment, the processor 360 may control the sensor module 330.

According to an embodiment, the sensor module 330 may measure a first impedance Z12 (or a first impedance value) corresponding to the first electrode unit 310 and a second impedance Z34 (or a second impedance value) corresponding to the second electrode unit 320. For example, the first impedance Z12 and the second impedance Z34 may mean impedances or contact impedances generated as the first electrode unit 310 and the second electrode unit 320 contact the user's body portion (e.g., the user's skin). Further, the first impedance Z12 and the second impedance Z34 may mean a contact impedance generated by the first electrode unit 310 and at least one of foreign bodies on the user's body portion (e.g., the user's skin), saliva, dead skin cells, skin, and skin portions, which contacts the second electrode unit 320.

According to an embodiment, the sensor module 330 may control the contact impedance for the first electrode unit 310 and the second electrode unit 320 before measurement for the user's biometric signal starts. For example, the sensor module 330 may measure the first impedance Z12 via the first electrode unit 310 and the second impedance Z34 via the second electrode unit 320. The sensor module 330 may control impedance so that the difference between the first impedance Z12 and the second impedance Z34 meets a designated range. For example, the designated range may mean a range in which the first impedance Z12 and the second impedance Z34 are identical to each other or are similar enough to make no difference therebetween so that no or little difference occurs between the first impedance Z12 and the second impedance Z34.

In the disclosure, for ease of description, when the first impedance Z12 and the second impedance Z34 are said to have the ‘same’ value or to have ‘no difference’ therebetween, this may mean that the difference between the first impedance Z12 and the second impedance Z34 meets the designated range. In other words, the processor 360 may control the circuit associated with adjustment of impedance, included in the electronic device 301 so that the difference between the first impedance Z12 and the second impedance Z34 meets the designated range.

The sensor module 330 may control the circuit associated with adjustment of impedance, included in the electronic device 301 so that the first impedance Z12 and the second impedance Z34 have the same value. For example, the electronic device 301 may control the state of connection of at least one of at least one element (e.g., a capacitor or resistor) connected with the first electrode unit 310 or at least one element (e.g., a capacitor or resistor) connected with the second electrode unit 320 so that the first impedance Z12 and the second impedance Z34 have the same value. Or, the electronic device 301 may adjust the element value (e.g., capacitance or load) of at least one of at least one variable element (e.g., a variable capacitor or variable resistor) connected with the first electrode unit 310 or at least one variable element (e.g., a variable capacitor or variable resistor) connected with the second electrode unit 320 so that the first impedance Z12 and the second impedance Z34 have the same value. In the disclosure, control of impedance may mean that the impedance measured at the corresponding channel is controlled by controlling the circuit or element connected with the contact impedance (e.g., the first impedance Z12 or the second impedance Z34) as described above.

The sensor module 330 may control the impedance so that no difference occurs between the first impedance Z12 and the second impedance Z34 and then measure the biometric signal (BS). By so doing, the sensor module 330 may remove or reduce an error due to contact impedance between the first electrode unit 310 and the second electrode unit 320.

According to an embodiment, the sensor module 330 may include an impedance analyzer 340, an impedance controller 350, and an amplifier 355.

The impedance analyzer 340 may measure the first impedance Z12 corresponding to the first electrode unit 310 and the second impedance Z34 corresponding to the second electrode unit 320. For example, the impedance analyzer 340 may measure the first impedance Z12 generated in the area where the first portion of the user's body contacts the first electrode unit 310. Further, the impedance analyzer 340 may measure the second impedance Z34 generated in the area where the second portion of the user's body contacts the second electrode unit 320.

The impedance analyzer 340 may include a first analysis unit 341 and a second analysis unit 342. For example, the first analysis unit 341 may measure the first impedance Z12 via the first electrode unit 310 connected with the first channel CH1. The second analysis unit 342 may measure the second impedance Z34 via the second electrode unit 320 connected with the second channel CH2. The first analysis unit 341 may be connected with the first electrode unit 310, and the second analysis unit 342 may be connected with the second electrode unit 320. For example, the first analysis unit 341 and the second analysis unit 342 may be separated in parallel to prevent mutual interference. The first analysis unit 341 may control the first channel CH1 of the sensor module 330, and the second analysis unit 342 may control the second channel CH2 of the sensor module 330. For example, the first channel CH1 may mean a path along which the biometric module 330 (e.g., the first analysis unit 341) is connected with the first electrode unit 310, and the second channel CH2 may mean a path along which the biometric module 330 (e.g., the second analysis unit 342) is connected with the second electrode unit 320. The first channel CH1 and the second channel CH2 may be positioned in parallel with each other.

The impedance analyzer 340 may analyze the first impedance Z12 and the second impedance Z34. The impedance analyzer 340 may identify the resistance and capacitance components of the first impedance Z12 and the second impedance Z34.

The impedance analyzer 340 may generate a control signal for controlling the impedance based on the results of analysis of the first impedance Z12 and the second impedance Z34. The impedance analyzer 340 may transmit the control signal to the impedance controller 350. For example, the control signal may mean a signal to adjust the impedance of the impedance controller 350 so that no difference is made between the first impedance Z12 and the second impedance Z34. The control signal may include parameters for adjusting resistance and capacitance.

The impedance controller 350 may adjust impedance based on the control signal. The impedance controller 350 may adjust the resistance and capacitance values based on the parameters included in the control signal.

The impedance controller 350 may include at least one resistor and capacitor for adjusting each of resistance and capacitance. For example, the impedance controller 350 may include at least one resistor and capacitor corresponding to the first channel CH1 and at least one resistor and capacitor corresponding to the second channel CH2.

The impedance controller 350 may control (or adjust) the total impedance of each of the channels to be the same impedance (or impedance value) based on the control signal CS. For example, the impedance controller 350 may adjust the resistance and capacitance between the amplifier 355 and the first electrode unit 310 to which the biometric signal is input. In other words, the impedance controller 350 may adjust the resistance and capacitance corresponding to the first channel CH1 which connects the first electrode unit 310 and the amplifier 355. Further, the impedance controller 350 may adjust the resistance and capacitance between the amplifier 355 and the second electrode unit 320 to which the biometric signal is input. In other words, the impedance controller 350 may adjust the resistance and capacitance corresponding to the second channel CH2 which connects the second electrode unit 320 and the amplifier 355.

After the impedance is controlled (e.g., after the total impedance of each of the first channel CH1 and the second channel CH2 is controlled to be the same impedance (or impedance value)), the sensor module 330 may obtain body-related signals received via the first electrode unit 310 and the second electrode unit 320. The sensor module 330 may output the obtained body-related signals to the amplifier 355 via the impedance controller 350. For example, the body-related signals may include a first input signal obtained from the first electrode unit 310 and a second input signal obtained from the second electrode unit 320. The first input signal and the second input signal may mean signals input to the amplifier 355.

The amplifier 355 may amplify the body-related signals output from the impedance controller 350 and generate a biometric signal BS. For example, the amplifier 355 may receive the first input signal and the second input signal and amplify, or differentially amplify, the first input signal and the second input signal. For example, the amplifier 355 may differentially amplify the first input signal and the second input signal and output the biometric signal BS. Further, the amplifier 355 may remove common components, e.g., noise, from the first input signal and the second input signal. In other words, the amplifier 355 may differentially amplify the input signals, remove the common components, and output the biometric signal BS which may be analyzed by the processor 360.

The processor 360 may analyze the biometric signal BS output from the amplifier 355. For example, the processor 360 may include a digital signal processor (DSP).

The processor 360 may analyze the biometric signal BS and provide the user's biometric information, body information, and/or health information according to the result of analysis. Further, the digital signal processor 360 may analyze the biometric signal BS and store the user's biometric information, body information, and/or health information according to the result of analysis.

Although FIG. 3 illustrates that the sensor module 330 includes separate components for ease of description, at least one of the components 340, 350, and 355 of the sensor module 330 may be implemented as a single component.

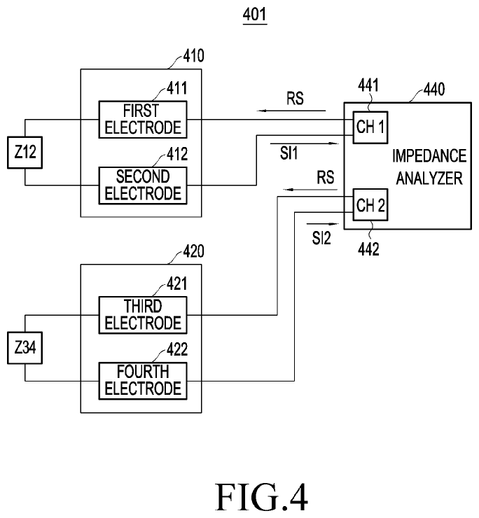

FIG. 4 is a block diagram illustrating operations of an electronic device according to various embodiments of the present invention.

Referring to FIG. 4, an electronic device 401 may be implemented in substantially the same or similar manner to the electronic device 301 described above in connection with FIG. 3. The first electrode unit 410 and the second electrode unit 420 may be implemented to be substantially the same or similar to the first electrode unit 310 and second electrode unit 320 of FIG. 3. Further, the impedance analyzer 440 may be implemented to be substantially the same or similar to the impedance analyzer 340 of FIG. 3.

FIG. 4 only illustrates the configuration related to the operation of measuring the impedances corresponding to the first electrode unit 310 and the second electrode unit 320, respectively, in the electronic device 301 of FIG. 3, for ease of description of the electronic device 401. However, the technical spirit of the disclosure is not limited thereto.

The impedance analyzer 440 may include a first analysis unit 441 and a second analysis unit 442. Before measuring biometric information, the impedance analyzer 440 may analyze the contact impedances corresponding to the first electrode unit 410 and the second electrode unit 420, respectively, via the first analysis unit 441 and the second analysis unit 442.

The first analysis unit 441 may output a reference signal (RS) to the first electrode unit 410. For example, the first analysis unit 441 may output a reference signal RS to the first electrode 411. The first analysis unit 441 may output the reference signal RS to the area (e.g., the user's first portion) contacted by the first electrode 411 and the second electrode 412, via the first electrode 411. The first analysis unit 441 may output the reference signal RS to the first electrode 411 while continuously or discretely varying frequency. For example, the contacted area (e.g., the user's first portion) may include the user's body portion (e.g., skin).

The second analysis unit 442 may output the reference signal RS to the second electrode unit 420. For example, the second analysis unit 442 may output the reference signal RS to the third electrode 421. The second analysis unit 442 may output the reference signal RS to the area (e.g., the user's second portion) contacted by the third electrode 421 and the fourth electrode 422, via the third electrode 421. The second analysis unit 442 may output the reference signal RS to the third electrode 421 while continuously or discretely varying frequency. For example, the contacted area (e.g., the user's second portion) may include the user's body portion (e.g., skin).

The reference signal RS may mean a signal output to the first electrode unit 410 and the second electrode unit 420 to measure the first impedance Z12 and the second impedance Z34 corresponding to the first electrode unit 410 and the second electrode unit 420. The reference signal RS may include signals with various frequencies.

The second electrode 412 may receive a first signal SI1 from the user's first portion. For example, the first signal SI1 may mean a signal which results as the reference signal RS is received by the second electrode 412 via (or passing through) the user's first portion. In other words, the first signal SI1 may mean the reference signal RS attenuated by the first impedance Z12.

The first analysis unit 441 may measure the first impedance based on the first signal SI1. For example, the first analysis unit 441 may determine a variation in frequency of the first signal SI1 according to a variation in frequency for the reference signal RS and measure the first impedance based on the frequency variation. Further, the first analysis unit 441 may determine a variation in frequency of the first signal SI1 according to a variation in frequency for the reference signal RS and determine the resistance component and capacitance component of the first impedance.

The fourth electrode 422 may receive a second signal SI2 from the user's second portion. For example, the second signal SI2 may mean a signal which results as the reference signal RS is received by the fourth electrode 422 via (or passing through) the user's second portion. In other words, the second signal S12 may mean the reference signal RS attenuated by the second impedance Z12.

The second analysis unit 442 may measure the second impedance based on the second signal S12. For example, the second analysis unit 442 may determine a variation in frequency of the second signal S12 according to a variation in frequency for the reference signal RS and measure the second impedance based on the frequency variation. Further, the second analysis unit 442 may determine a variation in frequency of the second signal S12 according to a variation in frequency for the reference signal RS and determine the resistance component and capacitance component of the second impedance.

According to various embodiments, the first analysis unit 441 may sequentially output a plurality of reference signals RS with different frequencies to the first electrode 411. For example, the first analysis unit 441 may continuously or discretely swap different frequencies (e.g., various frequencies ranging from DC to MHz) and output a plurality of reference signals RS.

Each of the plurality of reference signals RS may pass through the area contacted by the first electrode 411 and the area contacted by the second electrode 412. Further, each of the plurality of reference signals RS may be received by the second electrode 412 via the area contacted by the first electrode 411 and the area contacted by the second electrode 512.

The first analysis unit 441 may receive first signals SI1 individually corresponding to the plurality of reference signals RS via the second electrode 412. For example, the plurality of first signals SI1 may mean the reference signals RS attenuated via the impedance of the area contacted by the first electrode 411 and the impedance of the area contacted by the second electrode 412.

The first analysis unit 441 may determine the resistance component and capacitance component of the first impedance (e.g., the first impedance Z12 of FIG. 3) based on each of the first signals SI1. For example, the first impedance Z12 may include the impedance of the area contacted by the first electrode 411 and the impedance of the area contacted by the second electrode 412. In other words, the value of the first impedance Z12 may be the sum of the value of the impedance of the area contacted by the first electrode 411 and the value of the impedance of the area contacted by the second electrode 412.

According to various embodiments, since the respective capacitance components of the plurality of first signals SI1 received via the second electrode 412 are varied as the frequency of the reference signals RS with different frequencies, output to the first electrode 411, is varied, the first analysis unit 441 may identify a variation rate of each of the first signals SI1 received via the second electrode 412 corresponding to each of the reference signals RS with different frequencies. The first analysis unit 441 may identify the resistance component and capacitance of the first impedance Z12 based on the variation rate of each of the first signals SI1. Thus, the first analysis unit 441 may determine the first impedance (or the value of the first impedance).

According to various embodiments, the first analysis unit 441 may determine a frequency range of the plurality of reference signals RS output to the first electrode 411 according to the type of the biometric signal to be measured. For example, the first analysis unit 441 may determine a frequency range of the plurality of reference signals RS so that the ECG ranges from 0.01 Hz to 250 Hz, the EMG ranges from 25 Hz to 3,000 Hz, and the EEG ranges from 0.1 Hz to 100 Hz.

According to various embodiments, the second analysis unit 442 may sequentially output a plurality of reference signals RS with different frequencies to the third electrode 421. For example, the second analysis unit 442 may continuously or discretely swap different frequencies (e.g., various frequencies ranging from DC to MHz) and output a plurality of reference signals RS. Further, the second analysis unit 442 may output the same reference signals RS as the reference signals RS output to the first electrode 411 to the third electrode 521.

Each of the plurality of reference signals RS may pass through the area contacted by the third electrode 421 and the area contacted by the fourth electrode 422. Further, each of the plurality of reference signals RS may be received by the fourth electrode 422 via the area contacted by the third electrode 421 and the area contacted by the fourth electrode 422.

The second analysis unit 442 may receive second signals SI2 individually corresponding to the plurality of reference signals RS. For example, the plurality of second signals SI2 may mean the reference signals RS attenuated via the impedance of the area contacted by the third electrode 421 and the impedance of the area contacted by the fourth electrode 422.

The second analysis unit 442 may determine the resistance component and the capacitance component of the second impedance Z34 based on each of the second signals SI2. For example, the second impedance Z34 may include the impedance of the area contacted by the third electrode 421 and the impedance of the area contacted by the fourth electrode 422. In other words, the value of the second impedance Z34 may be the sum of the value of the impedance of the area contacted by the third electrode 421 and the value of the impedance of the area contacted by the fourth electrode 422.

According to various embodiments, since the respective capacitance components of the plurality of second signals SI2 received via the fourth electrode 422 are varied as the frequency of the reference signals RS with different frequencies, output to the third electrode 421, is varied, the second analysis unit 442 may identify a variation rate of each of the second signals SI2 received via the fourth electrode 422 corresponding to each of the reference signals RS with different frequencies. The second analysis unit 442 may identify the resistance component and capacitance of the second impedance Z34 based on the variation rate of each of the second signals SI2. Thus, the second analysis unit 442 may determine the first impedance (or the value of the first impedance).

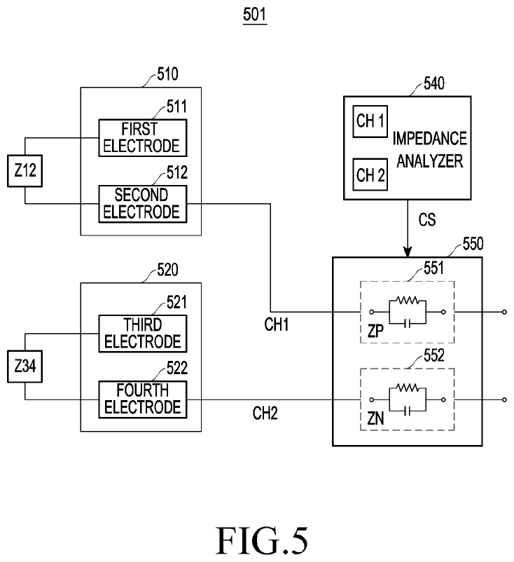

FIG. 5 is a block diagram illustrating operations of an electronic device according to various embodiments of the present invention.

Referring to FIG. 5, an electronic device 501 may be implemented in substantially the same or similar manner to the electronic device 301 of FIG. 3. The first electrode unit 510 and the second electrode unit 520 may be implemented to be substantially the same or similar to the first electrode unit 310 and second electrode unit 320 of FIG. 3. Further, the impedance analyzer 540 and the impedance controller 550 may be implemented in substantially the same or similar manner to the impedance analyzer 540 and impedance controller 550 of FIG. 3.

FIG. 5 only illustrates the configuration related to the operation of controlling the impedances corresponding to the first electrode unit 310 and the second electrode unit 320, respectively, in the electronic device 301 of FIG. 3, for ease of description of the electronic device 501. However, the technical spirit of the disclosure is not limited thereto.

The impedance analyzer 540 may determine the first impedance Z12 corresponding to the first channel CH1 and the second impedance Z34 corresponding to the second channel CH2 and may then transmit a control signal CS for controlling impedance to the impedance controller 550. For example, the control signal CS may mean a signal for the impedance controller 550 to adjust impedance so that the difference between the first impedance Z12 and the second impedance Z34 meets a designated range or there is no difference between the first impedance Z12 and the second impedance Z34. The control signal CS may include a parameter for the impedance controller 550 to adjust at least one of resistance and capacitance.

The impedance controller 550 may adjust impedance based on the control signal CS. The impedance controller 550 may adjust at least one of the resistance and capacitance based on the parameter included in the control signal CS.

The impedance controller 550 may include at least one element corresponding to each of a plurality of channels (e.g., the first channel CH1 and the second channel CH2) for controlling impedance. At least one element may include at least one of a resistor and a capacitor. For example, the impedance controller 550 may include at least one first element 651 corresponding to the first channel CH1 and at least one second element 652 corresponding to the second channel CH2. The impedance controller 550 may adjust the impedance value of the at least one first element 651 to ‘ZP’ and the impedance value of the at least one second element 652 to ‘ZN.’

According to various embodiments, at least one element included in the impedance controller 550 may be implemented as a variable element. The impedance controller 550 may control impedance by adjusting the variable element. Further, the impedance controller 550 may adjust the variable element, controlling impedance so that the total impedance of each of the first channel CH1 and the second channel CH2 is the same or meets a designated range.

According to various embodiments, at least one element included in the impedance controller 550 may be implemented as a circuit in which a plurality of elements is connected. The impedance controller 550 may control impedance by adjusting a plurality of elements. Further, the impedance controller 550 may adjust the variable element, controlling impedance so that the total impedance of each of the first channel CH1 and the second channel CH2 is the same or meets a designated range.

According to various embodiments, the impedance controller 550 may control impedance so that there is no difference between the first impedance Z12 corresponding to the first electrode unit 510 and the second impedance Z34 corresponding to the second electrode unit 520 or the difference between the first impedance Z12 and the second impedance Z34 meets a designated range. For example, the impedance controller 550 may control the first element 551 and the second element 552 so that the sum of the first impedance Z12 and ‘ZP’ and the sum of the second impedance Z34 and ‘ZN’ have the same impedance value (e.g., Z12+ZP=Z34+ZN).

According to various embodiments, the impedance controller 550 may control at least one element of the first element 551 and the second element 552. For example, if the first impedance Z12 is larger than the second impedance Z34, only the second element 552 corresponding to the second channel CH2 may be adjusted. Or, if the first impedance Z12 is smaller than the second impedance Z34, only the first element 551 corresponding to the first channel CH1 may be adjusted. Meanwhile, if the first impedance Z12 and the second impedance Z34 are the same (or substantially the same) (e.g., meet a designated range), the impedance controller 650 may refrain from adjusting the first element 551 and the second element 552.

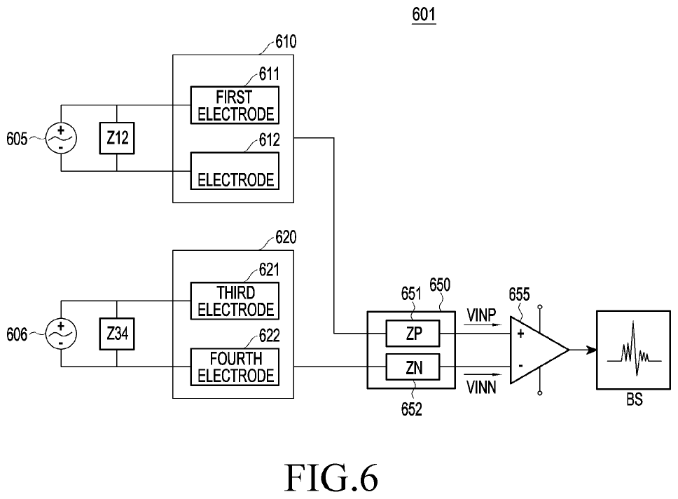

FIG. 6 is a block diagram illustrating operations of an electronic device according to various embodiments of the present invention.

Referring to FIG. 6, an electronic device 601 may be implemented in substantially the same or similar manner to the electronic device 301 of FIG. 3. The first electrode unit 610 and the second electrode unit 620 may be implemented to be substantially the same or similar to the first electrode unit 310 and second electrode unit 320 of FIG. 3. Further, the impedance analyzer 640, the impedance controller 650, and the amplifier 655 may be implemented in substantially the same or similar manner to the impedance analyzer 340, impedance controller 350, and amplifier 355 of FIG. 3.

FIG. 6 only illustrates the configuration related to the operation of controlling the impedances corresponding to the first electrode unit 310 and the second electrode unit 320 in the electronic device 301 of FIG. 3 and then generating a biometric signal BS, for ease of description of the electronic device 601. However, the technical spirit of the disclosure is not limited thereto.

After controlling impedance so that there is no difference between the first impedance Z12 and the second impedance Z34, the electronic device 601 may obtain body-related signals from the first electrode unit 610 and the second electrode unit 620.

According to various embodiments, the first electrode unit 610 and the second electrode unit 620 may obtain body-related signals from the user's first portion 605 and the user's second portion 606. Each of the body-related signals may be received by the amplifier 655 via the first electrode unit 610 or the second electrode unit 620 and the impedance controller 650. For example, a first input signal VINP may be received by a first terminal (e.g., a positive terminal) of the amplifier 655 via the first element 651 of the impedance controller 650 and the first electrode unit 610. For example, a second input signal VINN may be received by a second terminal (e.g., a negative terminal) of the amplifier 655 via the second element 652 of the impedance controller 650 and the second electrode unit 620. For example, the first input signal VINP and the second input signal VINN may be signals from which noise caused due to the difference between the first impedance Z12 and the second impedance Z34 has been removed.

The amplifier 655 may differentially amplify the first input signal VINP and the second input signal VINN and remove the common components. The amplifier 655 may output (or generate) a biometric signal BS resultant from differentially amplifying the first input signal VINP and the second input signal VINN and removing the common components. The amplifier 655 may transmit the biometric signal BS to a processor (e.g., the processor 360 of FIG. 3). The processor 360 may adjust the user's biometric signal BS, with the respective impedances of the first channel corresponding to the first electrode unit 610 and the second channel corresponding to the second electrode unit 620 adjusted.

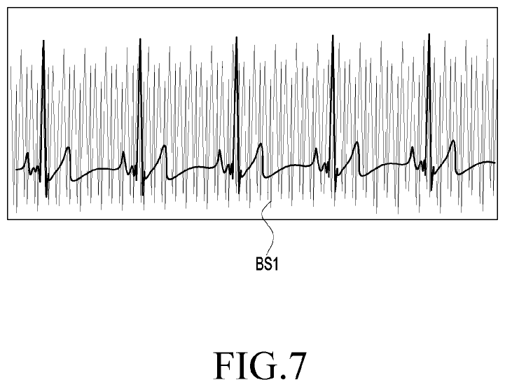

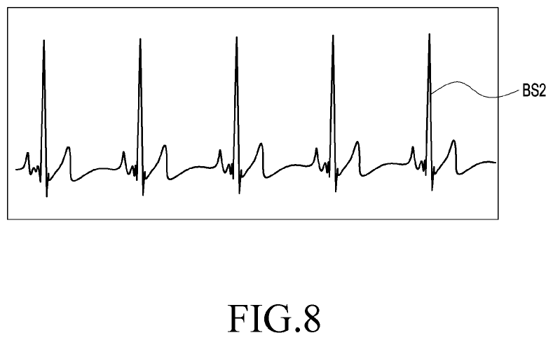

FIGS. 7 and 8 are views illustrating a biometric signal, from which noise caused by contact impedance has not been removed, and a noise-removed biometric signal, according to various embodiments of the present invention.

Referring to FIG. 7, a first biometric signal BS1 may mean a biometric signal obtained via a sensor module (e.g., the sensor module 330 of FIG. 3), without adjusting the difference between a first impedance corresponding to a first electrode unit (e.g., the first electrode unit 310 of FIG. 3) and a second impedance corresponding to a second electrode unit (e.g., the second electrode unit 320 of FIG. 3).

The first biometric signal B S1 contains lots of noise due to the difference between the first impedance and the second impedance. Further, it may be identified by referring to the first biometric signal BS1 that the noise caused due to the first impedance and the second impedance has also been amplified via the amplifier (e.g., the amplifier 355 of FIG. 3). Thus, as the difference between the first impedance and the second impedance increases, the first biometric signal BS1 may include more noise.

Referring to FIG. 8, a second biometric signal BS2 may mean a biometric signal obtained via the sensor module 330 after adjusting the difference between a first impedance corresponding to a first electrode unit (e.g., the first electrode unit 310 of FIG. 3) and a second impedance corresponding to a second electrode unit (e.g., the second electrode unit 320 of FIG. 3).

The second biometric signal BS2 does not contain the noise due to the difference between the first impedance and the second impedance. Or, the second biometric signal BS2 may contain a bit of noise due to the difference between the first impedance and the second impedance.

Thus, according to various embodiments of the present invention, the sensor module 330 may be controlled to make no difference between the first impedance and the second impedance or to allow the difference to meet a designated range, so that a noise-free or noise-reduced biometric signal may be obtained.

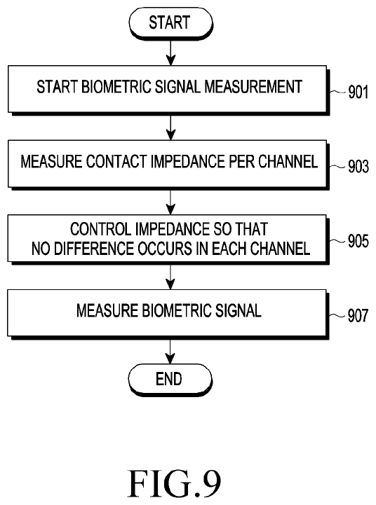

FIG. 9 is a flowchart illustrating operations of an electronic device according to various embodiments of the present invention.

Referring to FIG. 9, an electronic device (e.g., the electronic device 301 of FIG. 3) may start the operation of measuring a biometric signal in response to a command to request to measure a biometric signal (901). For example, if the user runs an application for measuring a biometric signal, the electronic device 301 may start the operation of measuring a biometric signal.

Before measuring a biometric signal, the electronic device 301 may perform the operation of compensating for at least one of contact impedances corresponding to channels of a sensor module (e.g., the sensor module 330 of FIG. 3).

The electronic device 301 may measure the contact impedance per channel of the sensor module 330 (903). For example, the electronic device 301 may measure the contact impedance of the area where the electrode unit connected with each of the channels of the sensor module 330 contacts the user's body portion. The electronic device 301 may measure the contact impedance for the electrode unit connected with each of the channels connected with the sensor module 330. The electronic device 301 may compare the contact impedances of the electrode units individually connected with the channels of the sensor module 330.

The electronic device 301 may control the impedance of the sensor module 330 so that no difference occurs between the respective contact impedances of the channels of the sensor module 330 (905). For example, the impedance controller (e.g., the impedance controller 350 of FIG. 3) of the sensor module 330 may control at least one resistor and capacitor for each of the channels included in the impedance controller 350. The impedance controller (e.g., the impedance controller 350 of FIG. 3) may control the at least one resistor and capacitor so that the total impedance of each of the channels has the same impedance value.

After the impedance of the sensor module 330 is controlled, the electronic device 301 may measure the biometric signal via the sensor module 330 (907). The electronic device 301 may obtain the biometric signal where the noise caused due to the difference in contact impedance between the electrode units has been removed.

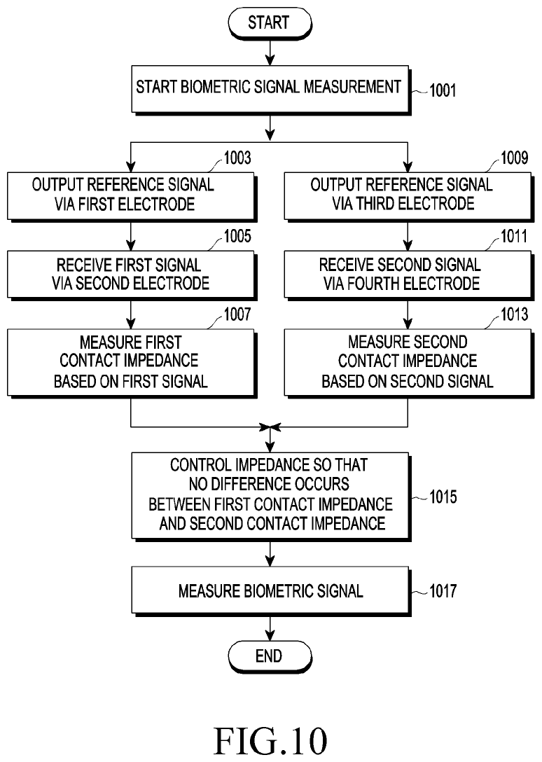

FIG. 10 is a flowchart illustrating operations of an electronic device according to various embodiments of the present invention.

Referring to FIG. 10, an electronic device (e.g., the electronic device 301 of FIG. 3) may start the operation of measuring a biometric signal in response to a command to request to measure a biometric signal (1001).

The electronic device 301 may output a reference signal RS via the first electrode 311 (1003). The electronic device 301 may sequentially output a plurality of reference signals RS with different frequencies to the first electrode 311. The electronic device 301 may continuously or discretely swap the different frequencies and output the plurality of reference signals RS to the first electrode 311.

The electronic device 301 may receive a first signal SI1 corresponding to the reference signal RS via the second electrode 312 (1005).

The electronic device 301 may measure a first contact impedance corresponding to the first electrode unit 310 based on the first signal SI1 (1007). For example, the electronic device 301 may measure the first contact impedance using an impedance meter (e.g., the impedance meter 340 of FIG. 3).

The electronic device 301 may output the reference signal RS via the third electrode 321 (1009). For example, the electronic device 301 may output the same reference signal RS as the first electrode 311 to the third electrode 321.

The electronic device 301 may receive a second signal SI2 corresponding to the reference signal RS via the fourth electrode 322 (1011).

The electronic device 301 may measure a second contact impedance corresponding to the second electrode unit 320 based on the second signal SI1 (1013). For example, the electronic device 301 may measure the second contact impedance using an impedance meter (e.g., the impedance meter 340 of FIG. 3).

The electronic device 301 may control the impedance so that no difference is made between the first contact impedance and the second contact impedance (1015). For example, the electronic device 301 may control impedance using an impedance controller (e.g., the impedance controller 350 of FIG. 3).

The electronic device 301 may control the impedance so that no difference is made between the first contact impedance and the second contact impedance and may then measure the biometric signal BS via the first electrode unit 310 and the second electrode unit 320 (1017). For example, the electronic device 301 may measure the biometric signal BS via at least one of the first electrode 311 and the second electrode and at least one of the third electrode 321 and the fourth electrode 322.

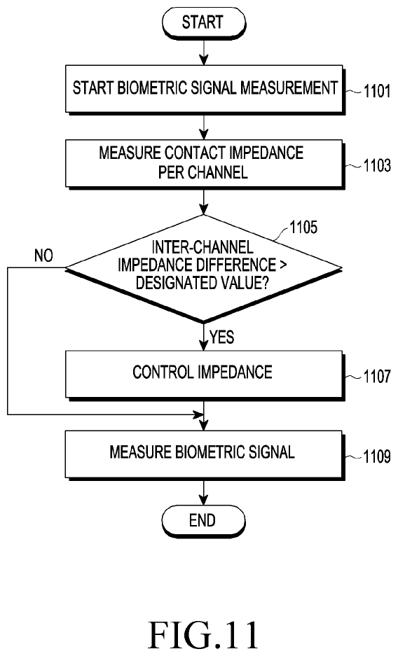

FIG. 11 is a flowchart illustrating operations of an electronic device according to various embodiments of the present invention.

Referring to FIG. 11, an electronic device (e.g., the electronic device 301 of FIG. 3) may start the operation of measuring a biometric signal in response to a command to request to measure a biometric signal (1101).

The electronic device 301 may measure the contact impedance per channel of the sensor module (e.g., the sensor module 330 of FIG. 3) (1103).

The electronic device 301 may determine the contact impedance of each channel of the sensor module 330. The electronic device 301 may determine the difference in impedance between the channels of the sensor module 330.

The electronic device 301 may compare the inter-channel impedance difference with a designated value (1105). For example, the designated value may mean a value where the impedance difference has substantially no influence on the biometric signal BS. The designated value may be set by the user or may be automatically set by a processor (e.g., the processor 360 of FIG. 3).

When the difference in impedance between the channels is larger than the designated value (yes in 1105), the electronic device 301 may control impedance using an impedance controller (e.g., the impedance controller 350 of FIG. 3). After controlling impedance, the electronic device 301 may measure the biometric signal BS via the first electrode unit 310 and the second electrode unit 320 (1109).

When the difference in impedance between the channels is identical to or smaller than the designated value (no in 1105), the electronic device 301 may refrain from controlling impedance using the impedance controller 350. Without controlling impedance, the electronic device 301 may measure the biometric signal BS via the first electrode unit 310 and the second electrode unit 320 (1109).

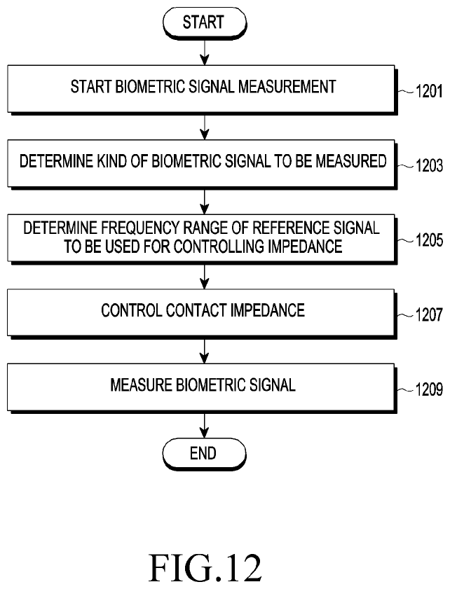

FIG. 12 is a flowchart illustrating operations of an electronic device according to various embodiments of the present invention.

Referring to FIG. 12, an electronic device (e.g., the electronic device 301 of FIG. 3) may start the operation of measuring a biometric signal in response to a command to request to measure a biometric signal (1201). For example, if the user runs an application for measuring a biometric signal, the electronic device 301 may start the operation of measuring a biometric signal.

The electronic device 301 may determine the type of a biometric signal to be measured (1203). For example, the electronic device 301 may determine that a biometric signal selected by the user is the biometric signal to be measured, on the execution screen of the application. Or, if the user runs the application for measuring the designated biometric signal, the electronic device 301 may determine that the designated biometric signal is the biometric signal to be measured.

The electronic device 301 may determine a frequency range of the reference signal RS for control of impedance, according to the type of the biometric signal to be measured (1205). The electronic device 301 may output a reference signal RS, in which a plurality of different frequencies have been sequentially varied, to the first electrode (e.g., the first electrode 311 of FIG. 3) and the third electrode (e.g., the third electrode 321 of FIG. 3), to determine the first contact impedance for the first electrode unit (e.g., the first electrode unit 310 of FIG. 3) and the second contact impedance for the second electrode unit (e.g., the second electrode unit 320 of FIG. 3). The electronic device 301 may determine the frequency range of the reference signal RS to be sequentially varied, depending on the type of the biometric signal to be measured.

The electronic device 301 may reduce the measurement time of the first contact impedance and the second contact impedance by outputting the reference signal RS to the first electrode 311 and the third electrode 321 according to the determined frequency range. In other words, although the frequency range of the reference signal RS is not limited to a specific frequency range, if the frequency range is limited according to the type of the biometric signal to be measured, the time of measurement of the contact impedance may be reduced.

The electronic device 301 may determine the first contact impedance and the second contact impedance and control impedance so that there is no difference between the first contact impedance and the second contact impedance (1207).

After controlling impedance, the electronic device 301 may measure the biometric signal BS (1209).

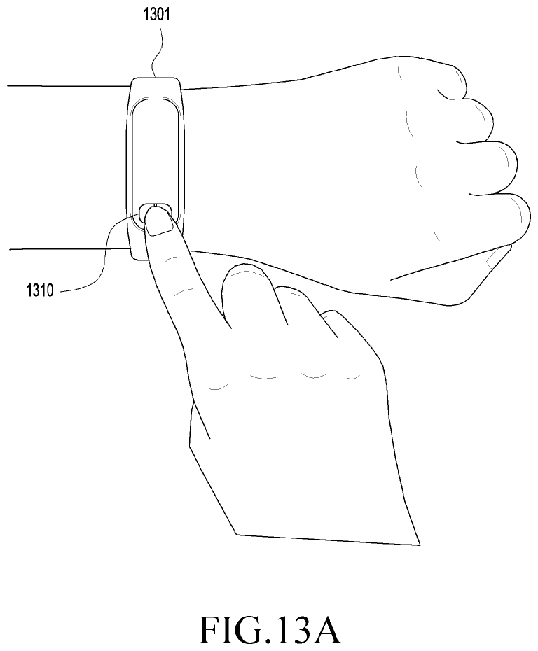

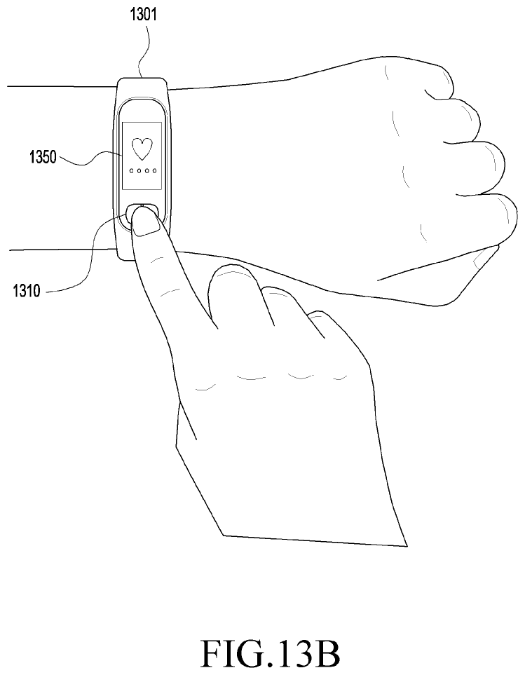

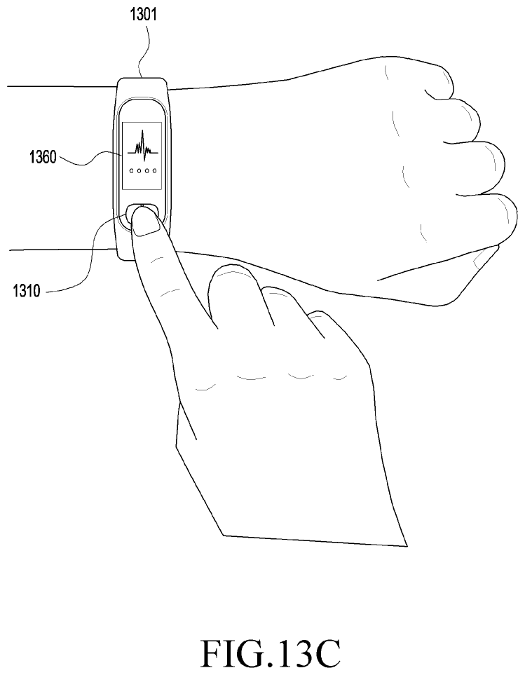

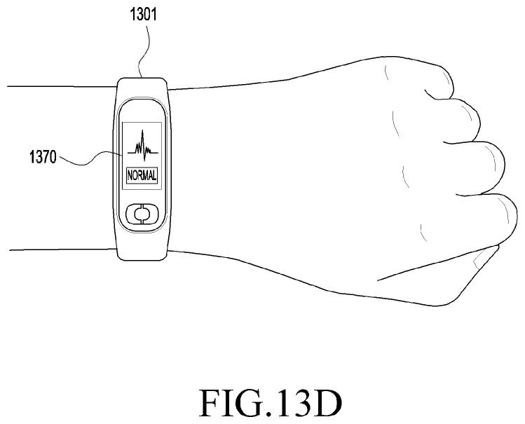

FIGS. 13A to 13D are views illustrating a user interface showing the operation of measuring a measurement by an electronic device according to various embodiments of the present invention.

Referring to FIGS. 13A to 13D, an electronic device 1301 may be implemented in substantially the same or similar manner to the electronic device 301 of FIG. 3.

Referring to FIG. 13A, the electronic device 1301 may start to measure a biometric signal according to the user's request (e.g., input). For example, the electronic device 1301 may execute an application related to measurement of biometric signal according to the user's input.

A first portion (e.g., a finger) of the user's body may contact a first electrode unit 1310, and a second portion (e.g., a wrist) of the user's body may contact a second electrode unit (e.g., the second electrode unit 320 of FIG. 3).

Referring to FIG. 13B, the electronic device 1301 may measure a first impedance (or first contact impedance), with the user's finger contacting the first electrode unit 1310. Further, the electronic device 1301 may measure a second impedance (or second contact impedance), with the user's wrist contacting the second electrode unit 320. Further, the electronic device 1301 may control the impedance so that no difference occurs between the first impedance and the second impedance.

The electronic device 1301 may measure the first impedance and the second impedance and, while controlling the impedance, display a first screen 1350. The first screen 1350 may show the state of preparing for measurement of a biometric signal.

Referring to FIG. 13C, after control of the impedance is done, the electronic device 1301 may measure the biometric signal, with the user's finger contacting the first electrode unit 1310 and the user's wrist contacting the second electrode unit 320.

The electronic device 1301 may display a second screen 1360 while measuring the biometric signal. The second screen 1360 may show the state of measuring the biometric signal.

Referring to FIG. 13D, the electronic device 1301 may analyze the measured biometric signal and provide the result of analysis to the user.

If analysis of the biometric signal is done, the electronic device 1301 may display a third screen 1370. The third screen 1370 may show the result of measurement of the biometric signal.

According to various embodiments of the present invention, an electronic device comprises a sensor module, a first electrode unit connected with a first channel of the sensor module, a second electrode unit connected with a second channel of the sensor module, and a processor configured to measure, via the sensor module, a first impedance between the first electrode unit and a first portion of a user's body contacting the first electrode unit and a second impedance between the second electrode unit and a second portion of the user's body contacting the second electrode unit, adjust, via the sensor module, impedances corresponding to the first channel and the second channel, respectively, so that a difference between the first impedance and the second impedance meets a designated range, based on the first impedance and the second impedance, and obtain, via the sensor module, the user's biometric signal, with the impedances of the first channel and the second channel adjusted.

The sensor module may include an impedance analyzer configured to measure the first impedance corresponding to the first electrode unit and the second impedance corresponding to the second electrode unit and output a control signal according to a result of the measurement and an impedance controller configured to control the impedances corresponding to the first channel and the second channel, respectively, in response to the control signal.

The impedance controller may include at least one resistor and capacitor for controlling the impedances corresponding to the first channel and the second channel, respectively.

The processor may be configured to, via the sensor module, output a reference signal via a first electrode of the first electrode unit and measure the first impedance based on a first signal which results as the reference signal is received via the first portion of the user's body by a second electrode of the first electrode unit and output the reference signal via a third electrode of the second electrode unit and measure the second impedance based on a second signal which results as the reference signal is received via the second portion of the user's body by a fourth electrode of the second electrode unit.

The processor may be configured to, via the sensor module, sequentially output a plurality of reference signals with different frequencies and determine resistance components and capacitance components of the first impedance and the second impedance based on a plurality of signals received individually corresponding to the plurality of reference signals.

The processor may be configured to determine a range in which the different frequencies of the reference signals are included, based on, at least, a type of the reference signal.

The processor may be configured to determine the difference between the first impedance and the second impedance via the sensor module and if the difference between the first impedance and the second impedance is larger than a designated value, adjust the impedances corresponding to the first channel and the second channel, respectively, so that the difference meets the designated range.

The processor may be configured to determine the difference between the first impedance and the second impedance via the sensor module and if the difference between the first impedance and the second impedance is equal to or smaller than a designated value, refrain from adjusting the impedances corresponding to the first channel and the second channel, respectively.

The electronic device may further comprise an amplifier configured to differentially amplify a first input signal obtained from the first electrode unit via the first channel and a second input signal obtained from the second electrode unit via the second channel and generate the biometric signal amplified.

The sensor module may be configured to sense at least one of the user's body fat, electrocardiogram, skin resistance, electromyogram, electroencephalogram, and electrooculogram.

According to various embodiments of the present invention, a method for operating an electronic device comprises measuring a first impedance between a first electrode unit connected with a first channel of a sensor module included in the electronic device and a first portion of a user's body contacting the first electrode unit, determining a second impedance between a second electrode unit connected with a second channel of the sensor module and a second portion of the user's body contacting the second electrode unit, adjusting impedances corresponding to the first channel and the second channel, respectively, so that a difference between the first impedance and the second impedance meets a designated range, based on the first impedance and the second impedance, and obtaining, via the sensor module, the user's biometric signal, with the impedances of the first channel and the second channel adjusted.

Setting the impedances corresponding to the first channel and the second channel, respectively, to be equal to each other may include outputting a control signal according to a result of measurement of the first impedance and the second impedance and controlling the impedances corresponding to the first channel and the second channel, respectively, in response to the control signal.

Controlling the impedances corresponding to the first channel and the second channel, respectively, may include controlling at least one resistor and capacitor included in each of the first channel and the second channel included in the sensor module.

Measuring the first impedance may include outputting a reference signal via a first electrode of the first electrode unit and measuring the first impedance based on a first signal which results as the reference signal is received via the first portion of the user's body by a second electrode of the first electrode unit, and measuring the second impedance may include outputting the reference signal via a third electrode of the second electrode unit and measuring the second impedance based on a second signal which results as the reference signal is received via the second portion of the user's body by a fourth electrode of the second electrode unit.

The method of operating the electronic device may further comprise sequentially outputting a plurality of reference signals with different frequencies and determining resistance components and capacitance components of the first impedance and the second impedance based on a plurality of signals received individually corresponding to the plurality of reference signals.

Varying and outputting the frequency of the reference signal may include determining a range in which the different frequencies of the reference signals are included, based on, at least, a type of the reference signal.

The method of operating the electronic device may further include determining the difference between the first impedance and the second impedance and, if the difference between the first impedance and the second impedance is larger than a designated value, setting the impedances corresponding to the first channel and the second channel, respectively, to be equal to each other so that the difference meets the designated range.

The method of operating the electronic device may further include determining the difference between the first impedance and the second impedance and, if the difference between the first impedance and the second impedance is equal to or smaller than a designated value, obtaining the biometric signal without adjusting the impedances corresponding to the first channel and the second channel, respectively.

In the method of operating the electronic device, obtaining the biometric signal may further include differentially amplifying a first input signal obtained from the first electrode unit via the first channel and a second input signal obtained from the second electrode unit via the second channel and obtaining the biometric signal.

The method of operating the electronic device may further comprise measuring the first impedance and the second impedance if measurement of the biometric signal is requested.

Each of the aforementioned components of the electronic device may include one or more parts, and a name of the part may vary with a type of the electronic device. The electronic device in accordance with various embodiments of the disclosure may include at least one of the aforementioned components, omit some of them, or include other additional component(s). Some of the components may be combined into an entity, but the entity may perform the same functions as the components may do.

The embodiments disclosed herein are proposed for description and understanding of the disclosed technology and does not limit the scope of the disclosure. Accordingly, the scope of the disclosure should be interpreted as including all changes or various embodiments based on the technical spirit of the disclosure.

1. An electronic device, comprising:
a sensor module;
a first electrode unit connected with a first channel of the sensor module;
a second electrode unit connected with a second channel of the sensor module; and
a processor configured to:
measure, via the sensor module, a first impedance between the first electrode unit and a first portion of a user's body contacting the first electrode unit and a second impedance between the second electrode unit and a second portion of the user's body contacting the second electrode unit;
adjust, via the sensor module, impedances corresponding to the first channel and the second channel, respectively, so that a difference between the first impedance and the second impedance meets a designated range, based on the first impedance and the second impedance; and
obtain, via the sensor module, a biometric signal of the user, with the impedances of the first channel and the second channel adjusted.

2. The electronic device of claim 1, wherein the sensor module includes:
an impedance analyzer configured to measure the first impedance corresponding to the first electrode unit and the second impedance corresponding to the second electrode unit and output a control signal according to a result of the measurement; and
an impedance controller configured to control the impedances corresponding to the first channel and the second channel, respectively, in response to the control signal.

3. The electronic device of claim 2, wherein the impedance controller includes at least one resistor and capacitor for controlling the impedances corresponding to the first channel and the second channel, respectively.

4. The electronic device of claim 1, wherein the processor is configured to, via the sensor module:
output a reference signal via a first electrode of the first electrode unit and measure the first impedance based on a first signal which results as the reference signal is received via the first portion of the user's body by a second electrode of the first electrode unit; and
output the reference signal via a third electrode of the second electrode unit and measure the second impedance based on a second signal which results as the reference signal is received via the second portion of the user's body by a fourth electrode of the second electrode unit.

5. The electronic device of claim 4, wherein the processor is configured to, via the sensor module:
sequentially output a plurality of reference signals with different frequencies and determine resistance components and capacitance components of the first impedance and the second impedance based on a plurality of signals received individually corresponding to the plurality of reference signals.

6. The electronic device of claim 5, wherein the processor is configured to determine a frequency range of the reference signals, based on, a type of the biometric signal.

7. The electronic device of claim 1, wherein the processor is configured to:
determine the difference between the first impedance and the second impedance via the sensor module; and
if the difference between the first impedance and the second impedance is larger than a designated value, adjust the impedances corresponding to the first channel and the second channel, respectively, so that the difference meets the designated range.

8. The electronic device of claim 1, wherein the processor is configured to:
determine the difference between the first impedance and the second impedance via the sensor module; and
if the difference between the first impedance and the second impedance is equal to or smaller than a designated value, refrain from adjusting the impedances corresponding to the first channel and the second channel, respectively.

9. The electronic device of claim 1, further comprising an amplifier configured to differentially amplify a first input signal obtained from the first electrode unit via the first channel and a second input signal obtained from the second electrode unit via the second channel and generate the biometric signal amplified.

10. The electronic device of claim 1, wherein the sensor module is configured to sense at least one of the user's body fat, electrocardiogram, skin resistance, electromyogram, electroencephalogram, and electrooculogram.

11. A method for operating an electronic device, the method comprising:
measuring a first impedance between a first electrode unit connected with a first channel of a sensor module included in the electronic device and a first portion of a user's body contacting the first electrode unit;
determining a second impedance between a second electrode unit connected with a second channel of the sensor module and a second portion of the user's body contacting the second electrode unit;
adjusting impedances corresponding to the first channel and the second channel, respectively, so that a difference between the first impedance and the second impedance meets a designated range, based on the first impedance and the second impedance; and
obtaining, via the sensor module, the user's biometric signal, with the impedances of the first channel and the second channel adjusted.

12. The method of claim 11, wherein setting the impedances corresponding to the first channel and the second channel, respectively, to be equal to each other includes:
outputting a control signal according to a result of measurement of the first impedance and the second impedance; and
controlling the impedances corresponding to the first channel and the second channel, respectively, in response to the control signal.

13. The method of claim 12, wherein controlling the impedances corresponding to the first channel and the second channel, respectively, includes controlling at least one resistor and capacitor included in each of the first channel and the second channel included in the sensor module.

14. The method of claim 11, wherein measuring the first impedance includes outputting a reference signal via a first electrode of the first electrode unit and measuring the first impedance based on a first signal which results as the reference signal is received via the first portion of the user's body by a second electrode of the first electrode unit, and wherein
measuring the second impedance includes outputting the reference signal via a third electrode of the second electrode unit and measuring the second impedance based on a second signal which results as the reference signal is received via the second portion of the user's body by a fourth electrode of the second electrode unit.

15. The method of claim 14, further comprising:
sequentially outputting a plurality of reference signals with different frequencies; and
determining resistance components and capacitance components of the first impedance and the second impedance based on a plurality of signals received individually corresponding to the plurality of reference signals.
